(19) United States
(12) Patent Application Publication
(10) Pub. No.: US 2024/0041752 A1
(43) Pub. Date: Feb. 8, 2024

(54) IMPROVEMENTS IN OR RELATING TO ORGANIC COMPOUNDS

(71) Applicant: Givaudan SA, VERNIER (CH)

(72) Inventors: Nicolas COCITO ARMANINO, Baden (CH); Gerhard BRUNNER, Opfikon (CH); Sandro DOSSENBACH, Gossau (CH); Fabian KUHN, Volketswil (CH); Sandrine PIANACCI, Paris (FR)

(21) Appl. No.: 18/264,325

(22) PCT Filed: Dec. 6, 2021
(86) PCT No.: PCT/EP2021/084437

§ 371 Date: Aug. 4, 2023

(30) Foreign Application Priority Data
Feb. 4, 2021 (EP) 21155289.8

(51) Int. Cl. A61K 8/92; A61K 8/31; A61Q 13/00; A61K 8/22

(52) U.S. Cl. CPC A61K 8/922; A61K 8/31; A61Q 13/00; A61K 8/22; A61K 2800/30; A61K 2800/52; A61K 2800/87

(57) ABSTRACT

A method of suppressing or eliminating a malodour from a product comprising a bio-sourced ingredient and a container for holding and/or dispensing the ingredient, wherein the bio-sourced ingredient contains ocimene and wherein the method comprises at least one of the steps of preventing reaction between ocimene and the container; physically removing any malodour formed; or treating the product with a malodour-counteracting agent.

### FIELD OF THE INVENTION

The present invention is concerned with methods and products for suppressing or eliminating unwanted or undesirable malodours that may be generated as a result of interaction phenomena between a bio-sourced ingredient and the container in which it is held, or from which it is dispensed, during its preparation, processing, distribution, storage or use.

### BACKGROUND OF THE INVENTION

In the normal life-cycle of a consumer product, from its design through its processing, production, distribution, storage and ultimately its use by consumers, it will come into contact with many containers. Interaction phenomena between consumer products and containers are known and can manifest themselves in many ways. For example, it is known that the structural integrity a container can be compromised by the ingredients that it holds. Similarly, materials that leach from containers can spoil the contents of those containers. Accordingly, producers of consumer products are naturally concerned to anticipate and control any interaction phenomena that could damage containers, or otherwise lead to unwanted modifications of the taste, sight, smell or feel of their products.

Depending upon the industry sector for which they were designed, consumer products can include one or more fragrance ingredients, cosmetic active ingredients, flavour ingredients, ingredients that deliver a health, wellness or nutritional benefit, or a functional benefit, such as dyes and pigments, preservatives, anti-microbial agents, anti-oxidants, texturizers, emulsifiers or the like. Furthermore, with the trend for increasing the usage of more natural, sustainable and renewable ingredients in consumer products, it is increasingly common for many of these ingredients to be bio-sourced. A bio-sourced ingredient, as that term is used herein, refers to an ingredient that is extracted and/or derived from a natural source, such as plants, fungi, bacteria, algae or animal sources. The ingredient may be native, i.e. extracted unmodified from its natural state, or taken from its natural state and purified or even chemically modified. Bio-sourced ingredients are often complex substances containing many by-products in trace amounts, and this complexity can heighten the risk of increasingly unpredictable interaction phenomena between the ingredients and the containers used to hold or dispense them.

When formulating with bio-sourced ingredients in the preparation of consumer products, and particularly those consumer products intended for use in perfumery, cosmetics, and flavours, as well as the food & beverage, health, wellness & nutrition and pharma industries, there is an ongoing need to prevent, reduce or eliminate any undesirable organoleptic effects that might arise as a result of unpredictable interaction phenomena between bio-sourced ingredients and the containers in which they are held or from which they are dispensed.

### SUMMARY OF THE INVENTION

When formulating with bio-sourced ingredients, in particular in the preparation of products useful in the fields of perfumery, cosmetics, flavours, food & beverage, health & well-being, nutrition and pharmaceuticals, applicant observed a surprising emanation of a sulphurous malodour reminiscent of a fermented, beer-like off-note, despite the fact the off-note was not detectable in the bio-sourced ingredients, as such. Although transient, the smell of an unexpected off-note emanating from a product, for example, in its testing phase, or even when the product is opened by a consumer, could lead to a loss of its attractiveness.

After a complex and exhaustive analytical investigation the applicant was able to attribute the development of transient off-notes to the generation of certain thiol compounds. More particularly, the applicant discovered in a surprising manner that certain bio-sourced ingredients contain naturally-occurring malodour precursor compounds that, under particular conditions of containment, can undergo chemical reactions with containers to produce thiol compounds that are implicated in the generation of the sulphurous malodour.

This surprising discovery underlying the present invention enables the management, and in particular the suppression or elimination, of sulphurous malodours that may be generated throughout the entire life-cycle of bio-sourced ingredients or any product or composition containing them.

Accordingly, the invention provides in a first aspect a method of suppressing or eliminating a malodour from a product comprising a bio-sourced ingredient and a container for holding and/or dispensing the ingredient, wherein the bio-sourced ingredient contains ocimene and wherein the method comprises at least one of the following steps:
Preventing contact of ocimene with material in the container capable of reacting with the ocimene to generate a thiol compound associated with a sulphurous malodour;
Removing from the product any thiol compound generated as the result of the reaction of ocimene with a co-reactant in the container; and
Treating the product with a malodour-counteracting agent.

In a second aspect, the invention provides a product comprising a bio-sourced ingredient and a container for holding and/or dispensing the ingredient, wherein the bio-sourced ingredient contains ocimene and the product is treated with a malodour-counteracting agent.

In a third aspect, the invention provides a product comprising a bio-sourced ingredient and a container for holding and/or dispensing the ingredient wherein the bio-sourced ingredient contains ocimene and the container is free of any co-reactants that can react with ocimene to generate a thiol compound associated with a sulphurous malodour.

The details, examples and preferences provided in relation to any one or more of the stated aspects or embodiments of the present invention will be further described herein and apply equally to all aspects and embodiments of the present invention. Any combination of embodiments, examples and preferences described herein below in all possible variations thereof are encompassed by the present invention unless otherwise indicated herein, or otherwise clearly contradicted by context.

### DETAILED DESCRIPTION OF THE INVENTION

The present invention is based on the surprising discovery that certain bio-sourced ingredients may, as a result of their natural origins, contain ocimene that can under certain conditions react with materials commonly found in containers to form thiol compounds, for example prenylthiol or 2-methyl-3-furanthiol, that are associated with the emanation of sulphurous malodours.

The generation of these thiol compounds in perfumery, flavours, cosmetics, food & beverage, health, wellness & nutrition and pharma applications as a result of interaction phenomena between bio-sourced ingredients and the containers used to hold and/or dispense them, was not known in the prior art.

The term “container” as used herein refers to any vessel or any piping, connectors, conduits or dispensers used or associated therewith, which are used to receive, hold and/or dispense a bio-sourced ingredient during its preparation, processing, distribution, storage and use, and includes reaction vessels, drum containers, or intermediate bulk containers (IBCs), packaging, dispensers, applicators, droppers, pumps including lotion pumps and spray pumps. Containers can comprise closure means, sealing means or dispensing means, such as valves, gaskets and the like, to prevent leakage from mating surfaces.

The bio-sourced ingredients are useful in applications as diverse as perfumery, cosmetics, flavours, food & beverages, health, wellness & nutrition or pharmaceuticals, to provide an organoleptic effect including the creation, modification or improvement of flavour, taste, aroma, colour, texture and appearance, or to impart a functional effect such as nutrition, health, wellness, anti-microbial or anti-oxidant, preservation, texturization or emulsification.

The bio-sourced ingredients can be employed in a finished consumer product, that is, they can be incorporated in a finished product for sale to consumers for their own use or enjoyment. Alternatively, the bio-sourced ingredients can be incorporated in a composition that is intended to be processed by further production activity into a consumer product, such as a perfume composition that is intended to be further incorporated into a consumer product, such as a perfume, fabric-care or home-care product. More particularly, a bio-sourced ingredient can form part of a perfume composition, a flavour composition, a colourant composition, a health, wellness & nutrition composition, or a composition having a functional effect such as a preservative composition, anti-microbial composition, an anti-oxidant composition, a texturizer composition or an emulsifier composition, that may form a component part of any of the consumer products disclosed herein.

Particular categories of consumer products include those products for use in fine perfumery, personal care, household care, laundry care, air care, and cosmetic treatment, as well as food & beverage products, products for delivering a health, wellness or nutritional effect, nutraceutical products and pharmaceutical products.

Particular examples of such products include alcoholic or non-alcoholic fine perfumes, household care, personal care or laundry care products, such as liquid detergents, powder detergents, fabric softeners, shampoos, shower gels, liquid soaps, dish wash products, conditioners, cleaners, body lotions, body creams, air fresheners, in-wash fabric perfumes or scent boosters, fabric freshener sprays, hygiene or care products, in particular in the range of body and hair care, cosmetic products, flavoured products, selected from the group consisting of foodstuffs, semi-luxury foods, snack foods, beverages, oral care products (e.g. oral hygiene products) nutraceuticals and products claiming health, wellness & nutrition benefits, or pharmaceutical products.

The bio-sourced ingredients referred to herein contain ocimene. As the term is used herein, ocimene refers to alpha-ocimene, cis-beta ocimene, trans beta-ocimene, allo-ocimene, or to mixtures thereof.

The applicant has found that ocimene is present in many bio-sourced ingredients and that under certain conditions it can act as malodour precursor compounds that is capable of reacting with any co-reactants present in containers to generate thiol compounds that are associated with a sulphurous malodour. However, owing to the complexity of bio-sourced ingredients, other trace compounds found in or derived from bio-sourced ingredients may also act as malodour precursor compounds. Accordingly, although hereafter reference is made specifically to ocimene, the skilled person will understand that the reference to ocimene can apply equally to other malodour precursor compounds present in bio-sourced ingredients that are capable of forming thiols, including but not limited to those compounds referred to hereunder.

Other malodour precursor compounds may be characterized as C5-scaffolds including but not limited to the following structures, which may be found in bio-sourced ingredients, or which are produced when bio-sourced ingredients interact with co-reactants found in containers:

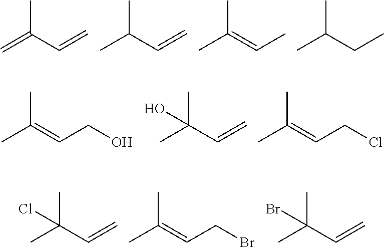

In accordance with the present invention, ocimene or other precursor compounds, such as the C5-scaffolds, have for the first time been identified as a materials displaying the propensity to produce thiol compounds implicated in the generation of sulphurous malodours under conditions referred to herein, and the invention resides in the surprising discovery of this property of ocimene and the means for either the prevention of interaction phenomena between it and co-reactants present in containers, or for the use of malodour-counteracting technology or other separation methods to suppress or eliminate the formation of the malodour. The co-reactants, which are described in more detail below, comprise vulcanized polymers or vulcanizing agents used in the preparation of such polymers. These vulcanizing agents and polymers are typically used in the formation of sealing means or dispensing means, such as gaskets used in containers, and particularly in valves, pumps and atomizers used in dispensers, and more particularly perfume dispensers. More particularly, these vulcanizing polymers and agents comprise sulphur, or sulphur-containing compounds.

There are many sources of ocimene in nature, and it is particularly abundant in many plants and fruits. Particular bio-sourced ingredients according to the invention that contain ocimene include, but are not limited to, the ingredient ocimene itself; Tagete Oil; Oppoponax Oil; Neroli Oil; Lavender Oil; Angelica Root Oil; Lavandin Abrialis Oil; Angelica Seed Oil; Lime Oxide; Fennel Oil; Menthe Citrate; Petitgrain Oil; Basil Oil; Lentisque Oil; Mint Oils, including Spearmint Oil And Peppermint Oil; Lime Oil; Lemon Oil; Orange Oil; Mandarin Oil; Ho Leaf; Hops; Kumquat; Mango; Bigarde; Parsley Oil; Asafoetida Oil; Ay Oil; Basil Oil; Bay Oil; Bergamot Oil; Black Pepper Oil; Buchu Oil; Cananga Oil; Carrot Seed Oil; Celery Seed Oil; Chamomile Oil; Cinnamon Bark Oil; Clary Sage Oil; Coriander Seed Oil; Cubebe Oil; Davana Oil; Dill Seed Oil; Estragon Oil; Eucalyptus Oil; Fennel Oil; Geraniol Oil; Ginger Oil; Grapefruit Oil; Jasmin Absolute; Laurel Leaf Oil; Lemon Oil; Lime Oil; Lovage Root Oil; Marjoram Oil; Neroli Oil; Niaouli Oil; Nutmeg Oil; Oil Of Labdanum; Orange Flower Oil; Palmarosa Oil; Pennyroyal Oil; Peppermint Terpenes; Petitgrain Mandarine Oil; Pine Needle Oil; Cardamom Oil; Rosemary Oil; Shiso Oil; Spearmint Oil; Star Anise Oil; Valerian Oil; Vanilla Extract; and mixture thereof.

Regarding the mechanism of thiol compound generation, the applicant believes, although does not intend to be bound by any theory, that prenylthiol formation from ocimene may proceed according to a process whereby oxidative or photolytic degradation products of ocimene react with hydrogen sulphide or carbon disulphide to form prenylthiol directly, according to the mechanism provided below. Oxidation of ocimene with oxygen (possibly in the presence of light) leads to formation of a peroxide, which undergoes Bayer-Villiger-like rearrangement to an oxonium compound. This is then hydrolysed to an aldehyde, which can undergo H-atom abstraction by a free radical (here a thiol radical) with concomitant decarbonylation to give the prenyl radical. This parallels the process observed in the natural generation of prenylthiol in beer from isohumulone. The prenyl radical can then trap thio-ethers to give prenylthiol and regenerate a thiol free radical catalyst.

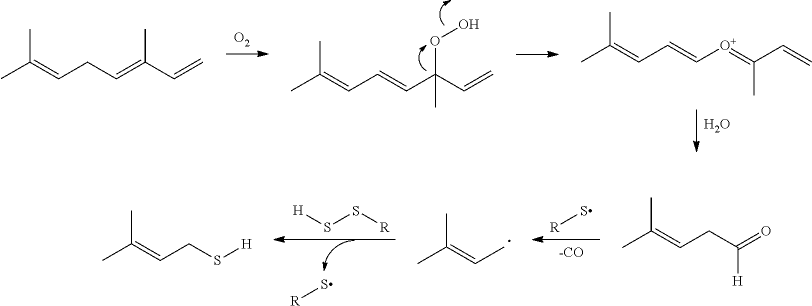

Alternatively, oxidative or photolytic degradation products of ocimene can react with hydrogen sulphide or carbon disulphide to form a xanthate, which can rearrange to form prenylthiol:

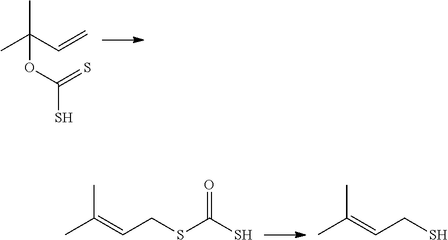

Another possible route involves the reaction of ocimene with the vulcanizing agent S₂Cl₂ in a radical chain reaction via a radical species to give an intermediate that can undergo alkyl migration to provide a cation, followed by 1,5-H shift to provide an intermediate compound, which can be hydrolysed to give prenylthiol:

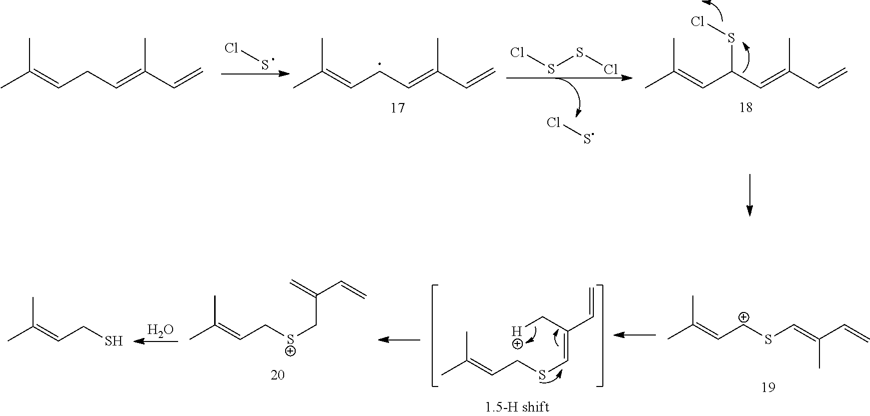

As for the malodorous thiol, 2-methyl-3-furanthiol, it may be produced according to the following reaction scheme:

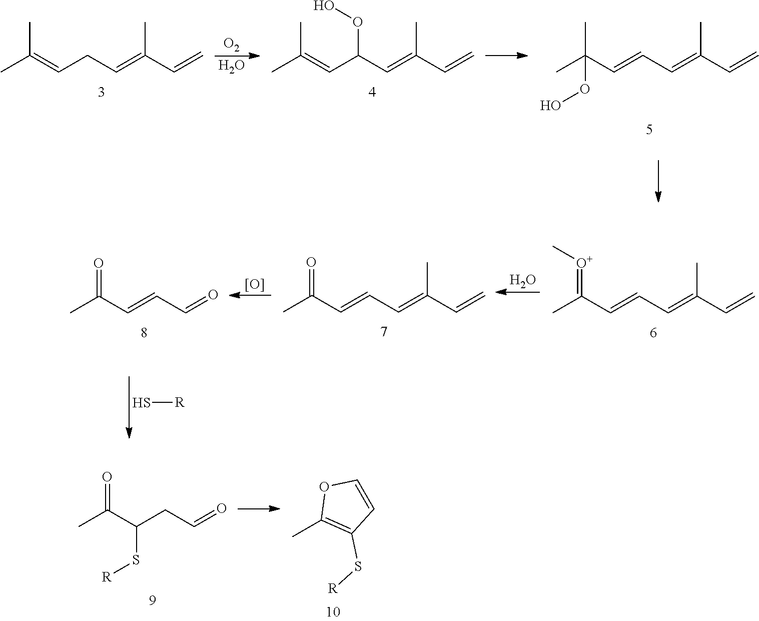

In addition to the role that ocimene plays in the generation of thiol compounds, after its oxidation in air to a peroxide it can become a strong electrophile, trapping thiols (possibly under acid catalysis) and acting as a reservoir that can thermally or oxidatively release thiol compounds in a slow and sustained manner, exacerbating the malodour problem by contributing to its longevity.

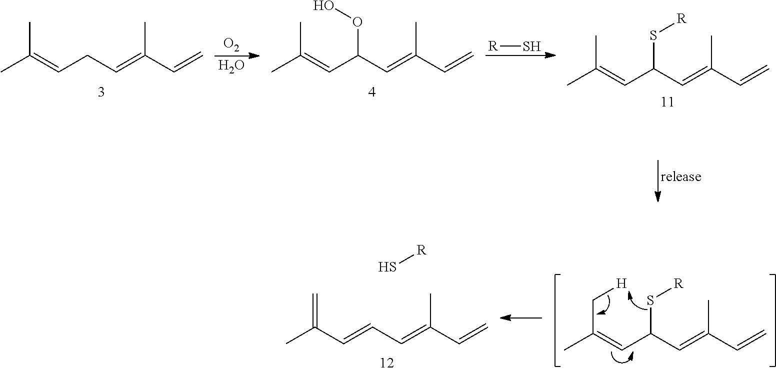

The bio-sourced ingredients referred to hereinabove, or more particularly the malodour precursor compounds they contain, most notably ocimene, have been shown to be a causative agent in the generation and release of thiol compounds, two of which have been identified for the first time.

However, in accordance with the teaching of the present invention, the sulphurous malodour associated with these thiol compounds can be managed, that is, it can be suppressed or eliminated.

Applicant has identified several means for suppressing or eliminating the sulphurous malodours, which means may be used singularly or in combination.

As stated above, in a particular aspect of the invention, malodour can be suppressed or eliminated by preventing the contact of ocimene with materials used in a container capable of reacting with ocimene to generate a thiol compound associated with a sulphurous malodour.

This can be achieved by selecting the container (or parts of the container) based on an understanding of the ingredients that will come into contact with it. That is to say, if a bio-sourced ingredient containing ocimene will be brought into contact with a container, then the container is selected such that it is free of any sulphur-containing vulcanized polymer or sulphur-containing vulcanizing agents used in the preparation of such a polymer.

Sulphur-containing vulcanized polymers include, but are not limited to, polyisoprene (latex), polybutadiene, butadiene-ethylene-styrene copolymer, styrene-isoprene copolymer, BUNA (butadiene-acrylonitrile copolymer), BUNA-S (butadiene-styrene copolymer), BUTYL (isobutylene-isoprene copolymer); and EPDM (ethylene-propylene-diene), including dienes selected from ENB (ethylidene norbornene), DCPD (dicyclopentadiene) and VNB (vinyl norbornene). The vulcanizing agents are principally elemental sulphur and sulphur-containing compounds, such as sulphur-donating substances selected from compounds bearing disulphide groups, e.g. disulphur dichloride, or thiazoles, such as 2-mercaptobenzothiazole, thiurams, dithiocarbamates, xanthates and organic thioureas.

Accordingly, to the extent that the container comprises any plastics, rubber or polymer elements, they should not be vulcanized with sulphur-containing vulcanizing agents. Suitable materials include but are not limited to polymers vulcanized with peroxides, and non-vulcanized polymers such as polyethylene (PE); polypropylene (PP), polyethylene terephthalate (PET) and copolymers of PE and ethylene vinyl alcohol (EVOH).

Polyethylene (PE) in the context of the present invention, includes all of the different forms of PE classified by their differing density and branching, such as ultra-high-molecular-weight polyethylene (UHMWPE), ultra-low-molecular-weight polyethylene (ULMWPE or PE-WAX), high molecular weight polyethylene (HMWPE), high density polyethylene (HDPE or PEAD), high-density cross-linked polyethylene (HDXLPE), cross-linked polyethylene (PEX or XLPE), medium-density polyethylene (MDPE), linear low-density polyethylene (LLDPE), low-density polyethylene (LDPE), very-low-density polyethylene (VLDPE), chlorinated polyethylene (CPE), preferably HDPE or LDPE. Similarly, polypropylene (PP), in the context of the present invention, also includes all of the different possible types of PP such as isotactic PP, syndiotactic PP, atactic PP, high crystalline PP (HcPP), random copolymers (RACO) of PP, block copolymers (HECO) of PP, uniaxially or biaxially oriented PP (BOPP). Polyethylene terephthalate (PET), in the context of the present invention, includes the different types of PET, such as polyethylenterephthalate and polyethylenterephthalate glycol.

Further particular embodiments of the present invention relate to the methods, products and containers described herein, wherein the container is free of sulphur-containing vulcanized polymers or sulphur-containing vulcanizing agents.

Containers typically comprise mating surfaces that generally require sealing means, such as a gasket or a valve to prevent leakage. If a container or part of the container such as a gasket or valve does contain, or is formed from, a polymeric material then preferably it will be formed of polymers vulcanized with peroxides, non-vulcanized polymers such as polyethylene (PE), polypropylene (PP), polyethylene terephthalate (PET) and copolymers of PE and ethylene vinyl alcohol (EVOH), or mixtures thereof.

Accordingly, the invention provides in another aspect a product comprising a bio-sourced ingredient comprising ocimene and a container for holding and/or dispensing the ingredient, wherein the container, or part of the container such as a gasket or a valve, is free of any sulphur-containing vulcanized polymer or sulphur-containing vulcanizing agents.

In another aspect of the invention there is provided a product comprising a bio-sourced ingredient comprising ocimene, and a container for holding and/or dispensing the ingredient, wherein the container comprises sealing means or dispensing means, such as a gasket or a valve, wherein the sealing means or dispensing means is free of sulphur-containing vulcanized polymers or sulphur-containing vulcanizing agents.

In still another aspect of the invention there is provide a container adapted to hold and/or dispense a bio-sourced ingredient comprising ocimene, wherein the container is free of any sulphur-containing vulcanized polymer or sulphur-containing vulcanizing agents.

In particular embodiments of the invention, containers that are free of any sulphur-containing vulcanized polymer or sulphur-containing vulcanizing agents, may contain or be comprised of any of the plastic, rubber or polymer materials referred to hereinabove.

In particular embodiments of the invention, container sealing means or dispensing means, such as gaskets or valves, are formed from polyethylene (PE), polypropylene (PP), polyethylene terephthalate (PET) and copolymers of PE and ethylene vinyl alcohol (EVOH), or mixtures thereof.

The containers may be reaction vessels, drum containers, or intermediate bulk containers (IBCs), packaging, dispensers, applicators, droppers, pumps including lotion pumps and spray pumps, including atomizers, and in particular atomizers for expressing perfumery.

Ensuring that sulphur-containing vulcanized polymers or vulcanizing agents are not used in containers represents an effective means of preventing the undesirable reaction of ocimene with any potential co-reactants present in the container, and thereby prevents the formation of thiol compounds and associated sulphurous malodour.

As stated above, another means by which the sulphurous malodour can be suppressed or eliminated in accordance with the present invention is by removing from the product any thiol compound generated as the result of the reaction of ocimene with a co-reactant in the container.

Removal of thiol compounds from the product can be achieved by separation means such as distillation or aeration, according to methods generally known in the art.

Alternatively or additionally, thiol compounds can be removed by a washing step. In particular embodiments, the thiol compounds can be removed by washing in the presence of a strong base, for example, a 2M NaOH solution. Alternatively or additionally, washing with an aqueous metal salt solution can be effective, e.g. washing with copper sulphate, or other suitable metal salt.

A further means of suppressing or eliminating a malodour from a product in accordance with the present invention comprises the step of treating the product with a malodour-counteracting agent.

As used herein, treatment of the product with a malodour-counteracting agent, includes the step of mixing the malodour-counteracting agent with the bio-sourced ingredient; pre-treating the container, or parts of the container such as the sealing or dispensing means, with the malodour-counteracting agent before bringing the bio-sourced ingredient into contact with the container; or both.

Suitable malodour-counteracting agents include those that can eliminate or suppress malodour by chemical reaction; or those that mask malodour by superimposing it with a pleasant, stronger odour; or those that cause cross-adaptation by blocking the malodour olfactory receptors; or those that suppress malodour by creating a negative deviation in Raoult's law.

The malodour-counteracting agents can be used singularly or in combination. The type and amount of agent one employs can be selected by the skilled person in a straightforward manner on the basis of the nature and intensity of the malodour that needs to be managed as well as the regulatory framework or the customer norms associated with the product into which the bio-sourced ingredient will be incorporated.

The malodour-counteracting agent will typically be mixed with the product containing the bio-sourced ingredient. However, additionally, or alternatively, if the container comprises a vulcanized polymer or vulcanizing agents, the malodour-counteracting agent can be incorporated into the vulcanized polymer, or the container can be treated with a malodour-counteracting agent before bringing the bio-sourced ingredient into contact with it.

In another aspect of the present invention, there is provided a product comprising ocimene-containing bio-sourced ingredient, a malodour-counteracting agent, and a container for holding and/or dispensing the same, wherein the malodour-counteracting agent is selected from the group consisting of an agent that can eliminate or suppress malodour by chemical reaction with the thiol compound causing the malodour; an agent that can mask the malodour by superimposing the malodour with a pleasant, stronger odour; an agent that causes cross-adaptation by blocking the malodour olfactory receptors; an agent that suppresses malodour by creating a negative deviation in Raoult's law; and an agent that can absorb malodour in a porous or cage-like structure.

Malodour-counteracting agents capable of chemically reacting with thiol compounds to eliminate or suppress malodour may be selected from metal ions, oxidising agents, such as peroxides, or non-odourant ingredients containing activated electrophilic groups that are capable of acting as Michael acceptors for thiol compounds.

Exemplary metal ion scavengers for thiol compounds include metal salts, such as zinc salts, and more particularly zinc carboxylates, e.g. zinc neodecanoate.

Exemplary oxidizing agents include any oxidant that can react with sulphur and/or thiols, such as peroxides, including hydrogen peroxide, organic peroxides, inorganic peroxides, peroxide salts, including all of their hydrated forms; oxidized halogen salts; oxidizing gases; per-acids, and oxidizing enzyme systems.

Organic peroxides include Tert-Butyl hydroperoxide (TBHP), Cumene hydroperoxide, 3-Chloroperbenzoic acid (mCPBA), Tert-butyl peroxide (Luperox® DI), tert-Butyl peracetate, tert-Butyl peroxybenzoate, Lauroyl peroxide (Luperox® LP), Benzoyl peroxide (Luperox® A75), Dicumyl peroxide 2,5-Bis(tert-butylperoxy)-2,5-dimethylhexane (Luperox® 101), 2,4-Pentanedione peroxide (Luperox® 224), tert-Butylperoxy 2-ethylhexyl carbonate (Luperox® TBEC), 1,1-Bis(tert-butylperoxy)cyclohexane (Luperox® 331M80), 2-Butanone peroxide and 1,1-Bis(tert-amylperoxy)cyclohexane.

Inorganic peroxides and metal-bound peroxides include oxone, alkali persulphates, vanadium peroxides, molybdenum peroxides and tungsten peroxides—all of which can be formed in situ by reaction of a peroxide source, such as hydrogen peroxide and a metal precursor.

Peroxide salts include sodium perborate (and hydrates), potassium peroxysulfate and Oxone®.

Oxidized halogen salts include bleach (sodium hypochlorite), calcium hypochlorite (and other hypochlorites), sodium chlorite, sodium chlorate and sodium perchlorate, as well as the hydrated forms of the foregoing. Other suitable cations can be employed instead of sodium, for example potassium.

Oxidizing gases include ozone, nitrous oxide and chlorine.

Per-acids include per-acetic acid, meta-chloro per-benzoic acid or magnesium mono-peroxy-phthalate.

Oxidizing enzyme systems include Laccases, Bilirubin Oxidases, Baeyer-Villiger Monooxigenases and Peroxidases.

The action of peroxides can be catalysed by number of transition metals, including Fe, Cu, Co, Ni, W, Mo as well as their salts and complexes.

The oxidizing gases and bleach can be generated in-situ from suitable precursors, e.g. by using electrolysis.

In preferred embodiments of the present invention the method of suppressing or eliminating malodour from a product comprising a bio-sourced ingredient and a container for holding and/or dispensing the ingredient, wherein the bio-sourced ingredient contains ocimene and wherein the container comprises a vulcanized polymer or sulphur-containing vulcanizing agents, comprises the step of treating the container, or parts of the container, with an oxidizing agent as described hereinabove.

The treatment step may consist of the pre-treatment of the container with the oxidizing agent before the bio-sourced ingredient is brought into contact with it, or the oxidizing agent can be mixed with the bio-sourced ingredient, or both. Preferably, however, the container is pre-treated with the oxidizing agent.

Pre-treatment of the container with an oxidizing agent can be carried out for an appropriate period of time sufficient to prevent or suppress malodour. This period of time can be elucidated using routine experimentation and is not limiting on the invention. Typically, the pre-treatment step can be carried out for a period up to about 24 hours, e.g. from about one hour up to about 24 hours, or any time increment in between. After pre-treatment, the container, or any part of the container treated, can be rinsed with an appropriate solvent, for example water and or ethanol.

The container must play a role in the formation of thiols since ocimene does not contain sulphur. Therefore, it is possible to conclude that the sulphur atoms necessary for the formation of thiols and malodour is present in the sulphur-containing vulcanized polymers or the sulphur-containing vulcanizing agents. For the sulphur to react with ocimene, it must be in the correct oxidation state. By pre-treating the container, it is possible to oxidize available sulphur atoms, which would otherwise be available to react with ocimene to create thiols. Similarly, treatment by mixing the oxidizing agent with the bio-sourced ingredient in the container will either oxidize available sulphur atoms in the container, such as elemental sulphur or sulphur-donating compounds selected from compounds bearing disulphide groups, or it will oxidize any thiol as it is formed.

Containers, as described herein that are free of sulphur atoms from elemental sulphur or sulphur-donating compounds, such as compounds containing disulphide groups, form an additional aspect of the present invention. Containers are considered to be free of such sulphur-containing material or compounds if there is no emanation of thiol malodour when the containers are contacted with bio-sourced ingredients containing ocimene.

The preferred oxidizing agent is hydrogen peroxide owing to its widely accepted use in industries including cosmetics, perfumery, food, medication and wellness. It is commercially available in a range of dilutions in water, for example 35 wt % in water.

Containers comprising vulcanized polymers or vulcanizing agents used in the preparation of said polymers, pre-treated with an oxidizing agent, and in particular hydrogen peroxide, represent a further aspect of the invention.

In particular embodiments of the invention the containers are selected from reaction vessels, drum containers, or intermediate bulk containers (IBCs), packaging, dispensers, applicators, droppers, pumps including lotion pumps and spray pumps, including atomizers for expressing perfumery or any of the other ingredients referred to herein.

In embodiments of the invention, the containers comprise closure means, sealing means or dispensing means, such as valves, gaskets and the like, to prevent leakage from mating surfaces.

Products comprising said pre-treated containers represent an additional aspect of the invention. More particularly, the products may be selected from a perfumery product, a home care product, an air care product, a personal care product, a cosmetic product, a flavour product, a food or beverage product, a product providing a health, wellness or nutrition benefit, an anti-microbial product, an anti-oxidant product, a preservative product, a colourant product, a texturizer product, an emulsifier product, a nutraceutical product or a pharmaceutical product.

Another class of malodour-counteracting agents capable of chemically reacting with thiol compounds to eliminate or suppress malodour is the class of non-odourant ingredients that can act as Michael acceptors. As the term is used herein, a non-odourant ingredient is an ingredient whose inclusion in a composition or product is not for the primary purpose of imparting a pleasant smell or aroma, but for the functional purpose of suppressing or eliminating malodour. Such compounds include aromatic unsaturated carboxylic esters, such as those disclosed in WO 02/751788, including dihexyl fumarate; alpha-beta unsaturated aldehyde, ketones or esters, as well as those ingredients in admixture with ammonium salts, such as those disclosed in WO 2012/126981; fumaric acid esters, such as those disclosed in U.S. Pat. No. 3,077,457; and esters of alpha-, beta-unsaturated monocarboxylic acids, such as those disclosed in U.S. Pat. No. 3,074,891. Any one or more of the foregoing ingredients are incorporated herein by reference as examples of suitable malodour counteracting agents.

In addition to these non-odourant ingredients, certain odourant ingredients, primarily of use as perfumery ingredients or for their aroma in flavour formulations are likewise known to be useful malodour-counteracting agents, either by interacting chemically with the sources of malodour to trap them, or by masking them with a pleasant smell. Exemplary classes of ingredients include those that contain a carbon-carbon double bond conjugated with one or more carbonyl groups. Aldehydes are the most commonly used materials of this class for malodour-counteractancy, such as trimethyl hexanal, other alkyl aldehydes, benzaldehyde, and vanillin; beta-dicarbonyl compounds, such as those disclosed in WO 2006/076821, including 2-(3,7-dimethyl-octa-2,6-dienylidene)-malonic acid diethyl ester; 3-octylidene-pentane-2,4-dione; 2-pyridin-2-ylmethylene-malonic acid diethyl ester; 2-octylidene-malonic acid dimethyl ester; 2-ethoxycarbonyl-but-2-enedioic acid diethyl ester; 2-acetyl-pent-2-enoic acid ethyl ester; 2-octylidene-malonic acid diethyl ester; 2-decylidene-malonic acid diethyl ester; 2-acetyl-dec-2-enoic acid ethyl ester; 2-(2-hydroxy-benzylidene)-1-phenyl-butane-1,3-dione; 2-(2-hydroxy-benzylidene)-malonic acid diethyl ester; 3-(2-hydroxy-benzylidene)-pentane-2,4-dione; 3-(2-hydroxy-4-methoxy-benzylidene)-pentane-2,4-dione; 2-(2-hydroxy-benzylidene)-3-oxo-butyric acid ethyl ester; 2-ethoxycarbonyl-oxymethylene-malonic acid diethyl ester; and 2-acetoxymethylene-malonic acid diethyl ester; ionones and other highly volatile odourant ingredients, such as those disclosed in U.S. Pat. No. 5,919,440, all of which foregoing classes and specific examples of ingredients are incorporated by reference as malodour-counteracting agents in accordance with the present invention.

Particular examples of preferred odourant ingredients useful as malodour counteracting agents are selected from the group consisting of Adoxal (2,6,10-trimethylundec-9-enal); Decylenic Aldehyde (2-decenal, 9-decenal, (E)-2-decenal, (Z)-2-decenal, (E)-4-decenal, (Z)-4-decenal, (E)-6-decenal, (Z)-6-decenal, (E)-7-decenal, or (Z)-7-decenal); Undecylic Aldehyde MOA (2-methyldecanal); Undecylenic Aldehyde (undecanal); Lauric Aldehyde (dodecanal); Lauric Aldehyde MNA (2-methylundecanal); Hexyl Aldehyde (hexanal); Heptyl Aldehyde (heptanal); Octyl Aldehyde (octanal); Nonyl Aldehyde (nonanal); Decyl Aldehyde (decanal); Benzaldehyde (benzaldehyde); Bourgeonal (3-(4-(tert-butyl)phenyl)propanal); Cyclal C (2,4-dimethylcyclohex-3-ene-1-carbaldehyde); Cyclamen Aldehyde (3-(4-isopropylphenyl)-2-methylpropanal); Cyclohexal (4-(4-hydroxy-4-methylpentyl)cyclohex-3-enecarbaldehyde); Dupical ((E)-4-((3a5,7aS)-hexahydro-1H-4,7-methanoinden-5(6H)-ylidene)butanal); Fennaldehyde (3-(4-methoxyphenyl)-2-methylpropanal); Florhydral (3-(3-isopropylphenyl)butanal); Hexenal-2 Trans ((E)-hex-2-enal); Hydratropic Aldehyde (2-phenylpropanal); Lilial (3-(4-(tert-butyl)phenyl)-2-methylpropanal); Mefranal (3-methyl-5-phenylpentanal); Melonal (2,6-dimethylhept-5-enal); Methyl Cinnamic Aldehyde ((Z)-2-methyl-3-phenylacrylaldehyde); Scentenal ((3aR,4R,65,7R,7aR)-6-methoxyoctahydro-1H-4,7-methanoindene-1-carbaldehyde); Para Tolyl Aldehyde (4-methyl-benzaldehyde); Tropional (3-(benzo[d][1,3]clioxo1-5-yl)-2-methylpropanal); Isononyl Aldehyde (3,5,5-trimethylhexanal); Citral ((E)-3,7-dimethylocta-2,6-dienal); Citronellal (3,7-dimethyloct-6-enal); Trans-2-Decenal ((E)-dec-2-enal); Dodecenal ((E)-dodec-2-enal); Ethyl Citral ((2E,6E)-3,7-dimethylnona-2,6-dienal); Hydroxycitronellal (7-hydroxy-3,7-dimethyloctanal); Isocyclocitral (2,4,6-trimethylcyclohex-3-enecarbaldehyde); Isovaleraldehyde (3-methylbutanal); Jasmorange (2-methyl-3-(4-methylphenyl)propanal); Mahonial ((4E)-9-hydroxy-5,9-dimethyl-4-decenal); Melonia (7-methoxy-3,7-dimethyloctanal); Methoxymelonal (6-methoxy-2,6-dimethylheptanal); Methyl lonal Beta ((Z)-2-methyl-4-(2,6,6-trimethyl-1-cyclohex-2-enyl)but-2-enal); Methyl Phenyl Hexenal (5-methyl-2-phenylhex-2-enal, (E)-5-methyl-2-phenylhex-2-enal, or (Z)-5-methyl-2-phenylhex-2-enal); Myraldene (4-(4-methylpent-3-en-1-yl)cyclohex-3-enecarbaldehyde); Nonadienal ((2E,6Z)-nona-2,6-dienal); Nonenal-6-Cis ((Z)-non-6-enal); Nympheal (3-(4-(2-methylpropyl)-2-methylphenyl)propanal); Oncidal ((E)-2,6,10-trimethylundeca-5,9-dienal); Phenoxy Acetaldehyde (2-phenoxyacetaldehyde); Phenyl Propionic Aldehyde (3-phenylpropanal); Precyclemone B (1-methyl-4-(4-methylpent-3-en-1-yl)cyclohex-3-enecarbaldehyde); Safranal (2,6,6-trimethylcyclohexa-1,3-dienecarbaldehyde); Shisolia (4-vinylcyclohex-1-enecarbaldehyde); Silvial (2-methyl-3-[4-(2-methylpropyl)phenyl]propanal); Syringa Aldehyde (2-(p-tolyl)acetaldehyde); Tetrahydro Citral (3,7-dimethyloctanal); Tricyclal (2,4-dimethylcyclohex-3-enecarbaldehyde); Trans-2-Tridecenal ((E)-tridec-2-enal); Trifernal (3-phenylbutanal); Cetonal (2-methyl-4-(2,6,6-trimethylcyclohex-2-en-1-yl)butanal); Cinnamic Aldehyde ((2E)-3-phenylprop-2-enal); Vernaldehyde (1-methyl-4-(4-methylpentyl)cyclohex-3-enecarbaldehyde); and mixtures thereof.

Other odourant ingredients that can be employed in the present invention may be selected from the group consisting of Acetal R ((2-(1-propoxyethoxy)ethyl)benzene); Acetanisole (1-(4-methoxyphenyl)ethanone); Acetivenol ((4,8-dimethyl-2-propan-2-ylidene-3,3a,4,5,6,8a-hexahydro-1H-azulen-6-yl) acetate); Acetoin (3-hydroxybutan-2-one); Acetophenone (1-phenylethanone); Agarbois (N-ethyl-N-(m-tolyl)propionamide); Agrumex (2-(tert-butyl)cyclohexyl acetate); Akigalawood ((3-pentyloxan-4-yl) acetate); Aldrone (2-methyl-4-(5,6,6-trimethylbicyclo[2.2.1]hept-2-yl-cyclohexanone); Alicate (2,6-dimethylheptan-4-yl acetate); Allyl Amyl Glycolate (prop-2-enyl 2-(3-methylbutoxy)acetate); Allyl Caproate (prop-2-enyl hexanoate); Allyl Cyclohexyl Propionate (prop-2-enyl 3-cyclohexylpropanoate); Allyl Oenanthate (prop-2-enyl heptanoate); Alpinone (1-(2,4,4,5,5-pentamethylcyclopent-1-en-1-yl)ethan-1-one); Amberketal (3,8,8,11a-tetramethyldodecahydro-1H-3,5a-epoxynaphtho[2,1-c]oxepine); Ambersage (2-methyl-2-(3-methylbutyl)-4,7-dihydro-1,3-dioxepine); Amyl Butyrate (pentyl butanoate); Amyl Caproate (pentyl hexanoare); Amyl Cinnamic Aldehyde ((Z)-2-benzylideneheptanal); Amyl Cinnamic Aldehyde Dimethyl Acetal ((Z)-2-benzylideneheptanal dimethylacetal); Amyl Phenyl Acetate (pentyl 2-phenylacetate); Anapear ((E)-methyl octa-4,7-dienoate); Anatolyl (phenylethyl 2-methylbutanoate); Anethole ((E)-1-methoxy-4-(prop-1-en-1-yl)benzene); Anther ((2-(isopentyloxy)ethyl)benzene); Aphermate (1-(3,3-dimethylcyclohexyl)ethyl formate); Azarbre (3,5-diethyl-2,5-dimethylcyclohex-2-enone); Azurone (7-isopentyl-2H-benzo[b][1,4]dioxepin-3(4H)-one); Benzophenone (diphenylmethanone); Benzyl Acetate; Benzyl Acetone (4-phenylbutan-2-one); Benzyl Butyrate (benzyl butanoate); Benzyl Cinnamate (benzyl 3-phenylprop-2-enoate); Benzyl Formate; Benzyl Isobutyrate (benzyl 2-methylpropanoate); Benzyl Isovalerate (benzyl 3-methylbutanoate); Benzyl Laurate (benzyl dodecanoate); Benzyl Methyl Ether ((methoxymethyl)benzene); Benzyl Phenyl Acetate (benzyl 2-phenylacetate); Benzyl Propionate (benzyl propanoate); Bergamyl Acetate (2-methyl-6-methyleneoct-7-en-2-yl acetate); Berryflor (ethyl 6-acetoxyhexanoate); Boisambrene Forte ((ethoxymethoxy)cyclododecane); Bornyl Acetate ((2S,4S)-1,7,7-trimethylbicyclo[2.2.1]heptan-2-yl acetate); Bornyl Acetate Liquid ((25,45)-1,7,7-trimethylbicyclo[2.2.1]heptan-2-yl acetate); Butyl Acetate; Butyl Butyrate (butyl butanoate); Butyl Butyro Lactate (1-butoxy-1-oxopropan-2-yl butanoate); Calypsone (6-methoxy-2,6-dimethyloctanal); Calyxol (ethyl 2-methyl-4-oxo-6-pentylcyclohex-2-ene-1-carboxylate); Camphor Synthetic ((1S,4S)-1,7,7-trimethylbicyclo[2.2.1]heptan-2-one); Carvone Laevo ((5R)-2-methyl-5-prop-1-en-2-ylcyclohex-2-en-1-one); Cashmeran (1,1,2,3,3-pentamethyl-2,3,6,7-tetrahydro-1H-inden-4(5H)-one); Cassione (4-(1-3-benzodioxol-5-yl)-2-buta none); Cedryl Acetate ((15,6R,8a R)-1,4,4,6-tetramethylocta hydro-1H-5,8a-methanoazulen-6-yl acetate); Celery Ketone (3-methyl-5-propylcyclohex-2-enone); Cepionate (methyl 2-(3-oxo-2-pentylcyclopentyl)acetate); Cetone V ((E)-1-(2,6,6-trimethylcyclohex-2-en-1-yl)hepta-1,6-dien-3-one); Cinnamyl Acetate ((E)-3-phenylprop-2-en-1-yl acetate); Cinnamyl Cinnamate (3-phenylprop-2-enyl 3-phenylprop-2-enoate); Cis Jasmone ((Z)-3-methyl-2-(pent-2-en-1-yl)cyclopent-2-enone); Cis-3-Hexenyl Acetate ((Z)-hex-3-enyl acetate); Cistulate (N,2-dimethyl-N-phenylbutanamide); Citral Diethyl Acetal (1,1-diethoxy-3,7-dimethylocta-2,6-diene); Citral Dimethyl Acetal ((E)-1,1-dimethoxy-3,7-dimethylocta-2,6-diene); Citrathal R ((Z)-1,1-diethoxy-3,7-dimethylocta-2,6-diene); Citronellol (3,7-dimethyloct-6-en-1-ol) Citronellyl Acetate (3,7-dimethyloct-6-en-1-yl acetate); Citronellyl Butyrate (3,7-dimethyloct-6-en-1-yl butanoate); Citronellyl Ethoxalate (3,7-dimethyloct-6-en-1-yl ethyl oxalate); Citronellyl Isobutyrate (3,7-dimethyloct-6-en-1-yl 2-methylpropanoate); Citronellyl Nitrile (3,7-dimethyloct-6-enenitrile); Citronellyl Propionate (3,7-dimethyloct-6-en-1-yl propanoate); Citronitrile (3-methyl-5-phenylpent-2-enenitrile); Civettone ((Z)-cycloheptadec-9-enone); Claritone (2,4,4,7-tetramethyloct-6-en-3-one); Coniferan (2-(tert-pentyl)cyclohexyl acetate); Corylone (2-hydroxy-3-methylcyclopent-2-enone); Cristalon (ethyl 2,6,6-trimethylcyclohexa-1,3-diene-1-carboxylate); Cuminyl Acetate ((4-isopropylphenyl)methyl acetate); Cyclogalbanate (allyl 2-(cyclohexyloxy)acetate); Cyclohexyl Ethyl Acetate (2-cyclohexylethyl acetate); Cyclopidene (methyl 2-yclopentylideneacetate); Cydrane (hexyl 2-methylbutanoate); Cyperate (3-tert-butyl-cyclohexyl acetate); Cyprisate (methyl 1,4-dimethylcyclohexanecarboxylate); Damascenone ((E)-1-(2,6,6-trimethylcyclohexa-1,3-dien-1-yl)but-2-en-1-one); Damascone Alpha ((E)-1-(2,6,6-trimethylcyclohex-2-en-1-yl)but-2-en-1-one); Damascone Beta ((E)-1-(2,6,6-trimethyl-1-cyclohexenyl)but-2-en-1-one); Decahydro Naphtyl Acetate Beta (1,2,3,4,4a,5,6,7,8,8a-decahydronaphthalen-2-yl acetate); Decahydro Naphtyl Formate Beta (1,2,3,4,4a,5,6,7,8,8a-decahydronaphthalen-2-yl formate); Decatone (6-isopropyloctahydronaphthalen-2(1H)-one); Decyl Acetate; Diethyl Malonate (diethyl propanedioate); Dihydro Ambrate (2-(sec-butyl)-1-vinylcyclohexyl acetate); Dihydro Anethole (1-methoxy-4-propylbenzene); Dihydro Farnesal ((Z)-3,7,11-trimethyldodeca-6,10-dienal); Dihydro Ionone Beta (4-(2,6,6-trimethylcyclohex-1-en-1-yl)butan-2-one); Dihydro Jasmone (3-methyl-2-pentylcyclopent-2-enone); Dihydro Myrcenyl Acetate (2,6-dimethyloct-7-en-2-yl acetate); Dimethyl Anthranilate (methyl 2-(methylamino)benzoate); Dimethyl Benzyl Carbinyl Acetate (2-methyl-1-phenylpropan-2-yl acetate); Dimethyl Benzyl Carbinyl Butyrate (2-methyl-1-phenylpropan-2-yl butanoate); Dimethyl Octenone (4,7-dimethyloct-6-en-3-one); Dimethyl Phenyl Ethyl Carbinyl Acetate (2-methyl-4-phenylbutan-2-yl acetate); Dimyrcetol (2,6-dimethyloct-7-en-2-ol); Dione (2-(2-(3,3,5-trimethylcyclohexyl)acetyl)cyclopentanone); Diphenyl Oxide (oxydibenzene); Dispirone (7,9-dimethylspiro[5.5]undecan-3-one); Dodecalactone Delta (6-heptyltetrahydro-2H-pyran-2-one); Dodecalactone Gamma (5-octyloxolan-2-one); Elintaal (3-(1-ethoxyethoxy)-3,7-dimethylocta-1,6-diene); Ethyl Acetate; Ethyl Amyl Ketone (octan-3-one); Ethyl Benzoate; Ethyl Butyrate (ethyl butanoate); Ethyl Cinnamate (ethyl 3-phenylprop-2-enoate); Ethyl Decadienoate ((2E,4Z)-ethyl deca-2,4-dienoate); Ethyl Hexanoate; Ethyl Isoamyl Ketone (6-methylheptan-3-one); Ethyl Isobutyrate (ethyl 2-methylpropanoate); Ethyl Isovalerate (ethyl 3-methylbutanoate); Ethyl Linalool ((E)-3,7-dimethylnona-1,6-dien-3-ol); Ethyl Linalyl Acetate ((Z)-3,7-dimethylnona-1,6-dien-3-yl acetate); Ethyl Maltol (2-ethyl-3-hydroxy-4H-pyran-4-one); Ethyl Methyl-2-Butyrate (ethyl 2-methylbutanoate); Ethyl Octanoate;Ethyl Oenanthate (ethyl heptanoate); Ethyl Pelargonate (ethyl nonanoate); Ethyl Phenyl Acetate (ethyl 2-phenylacetate); Ethyl Phenyl Glycidate (ethyl 3-phenyloxirane-2-carboxylate); Ethyl Propionate (ethyl propanoate); Ethyl Safranate (ethyl 2,6,6-trimethylcyclohexa-1,3-diene-1-carboxylate); Ethyl Trans-2-Octenoate (ethyl (E)-oct-2-enoate); Eucalyptol ((1s,4s)-1,3,3-trimethyl-2-oxabicyclo[2.2.2]octane); Eugenol (4-allyl-2-methoxyphenol); Fenchone Alpha (1,3,3-trimethylbicyclo[2.2.1]heptan-2-one); Fenchyl Acetate ((2S)-1,3,3-trimethylbicyclo[2.2.1]heptan-2-yl acetate); Fixolide (1-(3,5,5,6,8,8-hexamethyl-5,6,7,8-tetrahydronaphthalen-2-yl)ethanone); Floralate ((2,4-dimethylcyclohex-3-en-1-yl)methyl acetate); Floralozone (3-(4-ethylphenyl)-2,2-dimethylpropanal); Florane (2-heptyloxolane); Floridile ((E)-undec-9-enenitrile); Floropal (2,4,6-trimethyl-4-phenyl-1,3-dioxane); Folione (methyl oct-2-ynoate); Fraistone (ethyl 2-(2,4-dimethyl-1,3-dioxolan-2-yl)acetate); Freskomenthe (2-(sec-butyl)cyclohexanone); Gardenol (1-phenylethyl acetate); Gardocyclene ((3aR,6S,7aS)-3a,4,5,6,7,7a-hexahydro-1H-4,7-methanoinden-6-yl 2-methyl propanoate); Georgywood (1-(1,2,8,8-tetramethyl-1,2,3,4,5,6,7,8-octahydronaphthalen-2-yl)ethanone); Geranyl Acetone ((E)-6,10-dimethylundeca-5,9-dien-2-one); Geranyl Crotonate ((E)-3,7-dimethylocta-2,6-dien-1-yl but-2-enoate); Geranyl Formate ((E)-3,7-dimethylocta-2,6-dien-1-yl formate); Geranyl Isobutyrate ((E)-3,7-dimethylocta-2,6-dien-1-yl 2-methylpropanoate); Geranyl Phenyl Acetate ((E)-3,7-dimethylocta-2,6-dien-1-yl 2-phenylacetate); Geranyl Propionate ((E)-3,7-dimethylocta-2,6-dien-1-yl propanoate); Geranyl Propionate ((E)-3,7-dimethylocta-2,6-dien-1-yl propanoate); Geranyl Tiglate ((E)-(E)-3,7-dimethylocta-2,6-dien-1-yl 2-methylbut-2-enoate); Givescone (ethyl 2-ethyl-6,6-dimethylcyclohex-2-enecarboxylate); Guaiyl Acetate (2-(3,8-dimethyl-1,2,3,4,5,6,7,8-octa hydroazulen-5-yl)propan-2-yl acetate); Gyrane (2-butyl-4,6-dimethyl-3,6-dihydro-2H-pyran); Hedione (methyl 3-oxo-2-pentylcyclopentaneacetate); Heliotropyl Acetate (1,3-benzodioxol-5-ylmethyl acetate); Heptone (heptan-2-one); Herbanate ((2S)-ethyl 3-isopropylbicyclo[2.2.1]hept-5-ene-2-carboxylate); Herboxane (2-butyl-4,4,6-trimethyl-1,3-dioxane); Hexenyl Acetate Cis & Trans ((E)-hex-3-en-1-yl acetate); Hexenyl-3-Cis Butyrate ((Z)-hex-3-en-1-yl butanoate); Hexenyl-3-Cis Formate ((Z)-hex-3-en-1-yl formate); Hexenyl-3-Cis Hexenoate ((Z)-hex-3-en-1-yl (Z)-hex-3-enoate); Hexenyl-3-Cis Isobutyrate ((Z)-hex-3-en-1-yl 2-methylpropanoate); Hexenyl-3-Cis Methyl-2-Butyrate ((Z)-hex-3-en-1-yl 2-methyl butanoate); Hexenyl-3-Cis Propionate ((Z)-hex-3-en-1-yl propanoate); Hexenyl-3-Cis Tiglate ((Z)-hex-3-enyl] (E)-2-methylbut-2-enoate); Hexenyl-3-Trans Acetate ((E)-hex-3-enyl] acetate); Hexyl Acetate; Hexyl Butyrate (hexyl butanoate); Hexyl Isobutyrate (hexyl 2-methylpropanoate); Hexyl Methyl Butyrate/Cydrane (hexyl 2-methylbutanoate); Hexyl Propionate (hexyl propanoate); Homofuronol (2-ethyl-4-hydroxy-5-methylfuran-3(2H)-one); Hydratropic Aldehyde Dimethyl Acetal ((1,1-dimethoxypropan-2-yl)benzene); Hydroxycitronellal Dimethyl Acetal (8,8-dimethoxy-2,6-dimethyloctan-2-ol); Ionone Beta ((E)-4-(2,6,6-trimethylcyclohex-1-en-1-yl)but-3-en-2-one); Irisantheme ((E)-3-methyl-4-(2,6,6-trimethylcyclohex-2-en-1-yl)but-3-en-2-one); Irone Alpha ((E)-4-(2,5,6,6-tetramethylcyclohex-2-en-1-yl)but-3-en-2-one); Irone F ((E)-4-(2,5,6,6-tetramethylcyclohex-2-en-1-yl)but-3-en-2-one); Iso E Super (1-(2,3,8,8-tetramethyl-1,2,3,4,5,6,7,8-octahydronaphthalen-2-yl)etha none); Isoamyl Acetate (3-methylbutyl acetate); Isoamyl Butyrate (3-methylbutyl butanoate); Isoamyl Formate (3-methylbutyl formate); Isoamyl Propionate (3-methylbutyl propanoate); Isobornyl Propionate ((1S)-1,7,7-trimethylbicyclo[2.2.1]heptan-2-yl propanoate); Isobutyl Acetate (2-methylpropyl acetate); Isobutyl Isobutyrate (2-methylpropyl 2-methylpropanoate); Isobutyl Phenyl Acetate (2-methylpropyl 2-phenylacetate); Isodamascone (1-(2,4,4-trimethyl-1-cyclohex-2-enyl)but-2-en-1-one); Isojasmone (2-hexylcyclopent-2-enone); Isomenthone DI (2-isopropyl-5-methylcyclohexanone); Isononanyl Acetate (3,5,5-trimethylhexyl acetate); Isopentyl Isovalerate (3-methylbutyl 3-methylbutanoate); Isopentyrate (4-methylpent-4-en-2-yl 2-methylpropanoate); Isopropyl Methyl-2-Butyrate (isopropyl 2-methylbutanoate); Methyl Ionones ((1E)-1-(2,6,6-trimethylcyclohex-1-en-1-yl)pent-1-en-3-one; (1E)-1-(2,6,6-trimethylcyclohex-2-en-1-yl)pent-1-en-3-one; (3E)-3-methyl-4-(2,6,6-trimethylcyclohex-1-en-1-yl)but-3-en-2-one; (3E)-3-methyl-4-(2,6,6-trimethylcyclohex-2-en-1-yl)but-3-en-2-one), or mixtures thereof; Jasmacyclene ((3aR,6S,7aS)-3a,4,5,6,7,7a-hexahydro-1H-4,7-methanoinden-6-yl acetate); Jasmatone (2-hexylcyclopentanone); Jasmopyrane (3-pentyltetrahydro-2H-pyran-4-yl acetate); Jasverate ([(2E)-2-ethylidene-5-bicyclo[2.2.1]heptanyl] propanoate); Jessate (ethyl 2-acetyloctanoate); Karanal (5-(sec-butyl)-2-(2,4-dimethylcyclohex-3-en-1-yl)-5-methyl-1,3-dioxane); Kefarene (1-(4-methoxy-2,2,6,6-tetramethylcyclohex-3-en-1-yl)ethanone); Kephalis (4-(1-ethoxyethenyl)-3,3,5,5-tetramethylcyclohexan-1-one); Ketoisophorone (2,6,6-trimethylcyclohex-2-ene-1,4-dione); Koavone ((Z)-3,4,5,6,6-pentamethylhept-3-en-2-one); Labienone ((E)-2,4,4,7-tetramethylnona-6,8-dien-3-one); Leaf Acetal ((Z)-1-(1-ethoxyethoxy)hex-3-ene); Lemonile ((2E,6Z)-3,7-dimethylnona-2,6-dienenitrile); Linalyl Acetate (3,7-dimethylocta-1,6-dien-3-yl acetate); Linalyl Cinnamate (3,7-dimethylocta-1,6-dien-3-yl 3-phenylprop-2-enoate); Linalyl Formate (3,7-dimethylocta-1,6-dien-3-yl formate); Linalyl Isobutyrate (3,7-dimethylocta-1,6-dien-3-yl 2-methylpropanoate); Linalyl Propionate (3,7-dimethylocta-1,6-dien-3-yl propanoate); Magnolione (3-(2-oxopropyl)-2-pentylcyclopentan-1-one); Maltol (3-hydroxy-2-methyl-4H-pyran-4-one); Maltyl Isobutyrate (2-methyl-4-oxo-4H-pyran-3-yl 2-methylpropanoate); Manzanate (ethyl 2-methylpentanoate); Melusat (ethyl 3,5,5-trimethylhexanoate); Menthanyl Acetate (2-(4-methylcyclohexyl)propan-2-yl acetate); Menthone (2-isopropyl-5-methylcyclohexanone); Mercapto-8 Menthene-1 Para (2-(4-methylcyclohex-3-en-1-yl)propane-2-thiol); Metambrate ((2-butan-2-yl-1-methylcyclohexyl) acetate); Methyl Acetophenone (1-(p-tolyl)ethanone); Methyl Amyl Ketone (heptan-2-one); Methyl Camomille (butyl 2-methylpentanoate); Methyl Cedryl Ketone (1-((15,8aS)-1,4,4,6-tetramethyl-2,3,3a,4,5,8-hexahydro-1H-5,8a-methanoazulen-7-yl)etha none); Methyl Cinnamate (methyl 3-phenylprop-2-enoate); Methyl Corylone (3,4-dimethylcyclopentane-1,2-dione); Methyl Cyclogeranate (methyl 2,6,6-trimethylcyclohex-2-ene-1-carboxylate); Methyl Dihydro Isojasmonate (methyl 2-hexyl-3-oxocyclopentane-1-carboxylate); Methyl Epi Jasmonate ((Z)-methyl 2-(3-oxo-2-(pent-2-en-1-yl)cyclopentyl)acetate); Methyl Heptenone (6-methylhept-5-en-2-one); Methyl Hexanoate (methyl hexanoate); Methyl Hexyl Ketone (octan-2-one); Methyl Jasmonate (methyl 2-(3-oxo-2-pent-2-enylcyclopentyl)acetate); Methyl Linoleate ((9E,12E)-methyl octadeca-9,12-dienoate); Methyl Methyl-2 Butyrate (methyl 2-methylbutanoate); Methyl Nonyl Ketone (undecan-2-one); Methyl Pamplemousse (6,6-dimethoxy-2,5,5-trimethylhex-2-ene); Methyl Phenyl Acetate (methyl 2-phenylacetate); Methyl Undecylenate (methyl (E)-undec-9-enoate); Methylionantheme Gamma ((E)-3-methyl-4-(2,6,6-trimethylcyclohex-1-en-1-yl)but-3-en-2-one); Muscenone ((Z)-3-methylcyclopentadec-5-enone); Muscone (3-methylcyclopentadecanone); Myraldyl Acetate ((4-(4-methylpent-3-en-1-yl)cyclohex-3-en-1-yl)methyl acetate); Myrascone (ethyl 2-ethyl-3,6,6-trimethylcyclohexene-1-carboxylate); Myrcene (7-methyl-3-methyleneocta-1,6-diene); Myrcenyl Acetate ((2-methyl-6-methylideneoct-7-en-2-yl) acetate); Nectaryl (2-(2-(4-methylcyclohex-3-en-1-yl)propyl)cyclopentan-1-one); Neobergamate Forte (2-methyl-6-methyleneoct-7-en-2-yl acetate); Neofolione ((E)-methyl non-2-enoate); Nerolidyl Acetate ((Z)-3,7,11-trimethyldodeca-1,6,10-trien-3-yl acetate); Nerolione (1-(3-methylbenzofuran-2-yl)ethanone); Nerone (1-(2-methyl-5-propan-2-yl-1-cyclohex-2-enyl)propan-1-one); Neryl Acetate ((Z)-3,7-dimethylocta-2,6-dien-1-yl acetate); Nonadienyl Acetate ((2Z,6E)-2,6-nonadien-1-yl acetate); Nonanone-2 (nonan-2-one); Nonyl Acetate (nonyl acetate); Nootkatone Crystals (4,4a-dimethyl-6-(prop-1-en-2-yl)-4,4a,5,6,7,8-hexahydronaphthalen-2(3H)-one); Nopyl Acetate (2-(6,6-dimethylbicyclo[3.1.1]hept-2-en-2-yl)ethyl acetate); Octenyl Acetate (oct-1-en-3-yl acetate); Octyl Acetate; Octyl Aldehyde Dimethyl Acetal (octanal dimethyl acetal); Opalal (7-isopropyl-8,8-dimethyl-6,10-dioxaspiro[4.5]decane); Orivone (4-(tert-pentyl)cyclohexanone); Oxyoctaline Formate (2,4a,5,8a-tetramethyl-1,2,3,4,4a,7,8,8a-octahydronaphthalen-1-yl formate); Pandanol ((2-methoxyethyl)benzene); Para Butyl Cyclohexyl Acetate (4-(tert-butyl)cyclohexyl acetate); Para Cresyl Acetate ((4-methylphenyl) acetate); Para Cresyl Caprylate ((4-methylphenyl) octanoate); Para Cresyl Isobutyrate ((4-methylphenyl) 2-methylpropanoate); Para Cresyl Phenyl Acetate ((4-methylphenyl) 2-phenylacetate); Para Cymene (p-cymene); Para Methyl Benzyl Acetate ((4-methylphenyl)methyl acetate); Para Tert Butyl Cyclohexanone (4-(tert-butyl)cyclohexanone); Paradisamide (2-ethyl-N-methyl-N-(m-tolyl)butanamide); Parmavert (1,1-dimethoxynon-2-yne); Peach Pure (5-heptyldihydrofuran-2(3H)-one); Pelargene (2-methyl-4-methylene-6-phenyltetrahydro-2H-pyran); Pepperwood (3,7-dimethylocta-1,6-dien-3-yl dimethylcarbamate); Peranat (2-methylpentyl 2-methylpentanoate); Petiole (2-propan-2-yloxyethylbenzene); Phantolide (1-(1,1,2,3,3,6-hexamethyl-2H-inden-5-yl)ethanone); Pharaone (2-cyclohexylhepta-1,6-dien-3-one); Phenoxy Ethyl Isobutyrate (2-(phenoxy)ethyl 2-methylpropanoate); Phenyl Ethyl Alcohol (2-phenylethanol); Phenyl Ethyl Butyrate (2-phenylethyl butanoate); Phenyl Ethyl Cinnamate (2-phenylethyl 3-phenylprop-2-enoate); Phenyl Ethyl Formate (2-phenylethyl formate); Phenyl Ethyl Isobutyrate (2-phenylethyl 2-methylpropanoate); Phenyl Ethyl Isovalerate (2-phenylethyl 3-methylbutanoate); Phenyl Ethyl Phenyl Acetate (2-phenylethyl 2-phenylacetate); Phenyl Ethyl Tiglate (phenethyl (E)-2-methylbut-2-enoate); Phenyl Propyl Acetate (3-phenylpropyl acetate); Piconia ((8aR)-4,4,8,8-tetramethylhexahydro-1H-3,8a-methanonaphthalen-5(6H)-one); Pivacyclene ((3aR,6S,7aS)-3a,4,5,6,7,7a-hexahydro-1H-4,7-methanoinden-6-yl 2,2-dimethylpropanoate); Pivarose (2,2-dimethyl-2-pheylethyl propanoate); Plicatone ((4aS,8aR)-7-methyloctahydro-1,4-methanonaphthalen-6(2H)-one); Poirenate (ethyl 2-cyclohexylpropanoate); Pomarose ((2E,5E)-5,6,7-trimethylocta-2,5-dien-4-one); Precarone ((15,4R,65)-4,7,7-trimethyl-4-(3-methylbut-2-en-1-yl)bicyclo[4.1.0]heptan-3-one); Prenyl Acetate (3-methylbut-2-en-1-yl acetate); Prunolide (5-pentyldihydrofuran-2(3H)-one); Quintone (2-pentylcyclopentanone); Raspberry Ketone (4-(4-hydroxyphenyl)butan-2-one); Resedal (2-(cyclohexylmethyl)-4,4,6-trimethyl-1,3-dioxane); Rhodinyl Acetate (mixture of 3,7-dimethyloct-6-en-1-yl acetate and 3,7-dimethyloct-7-en-1-yl acetate); Rhubofix ((2 R,8aS)-3′,6-d imethyl-3,4,4a,5,8,8a-hexahydro-1 H-spiro[1,4-methanonaphthalene-2,2′-oxirane]); Rosacetol (2,2,2-trichloro-1-phenylethyl acetate); Rum Acetal (1,1-diethoxycyclohexane); Safraleine (2,3,3-trimethyl-1-indanone); Spirogalbanone Pure (1-(spiro[4.5]dec-6-en-7-yl)pent-4-en-1-one); Styrallyl Propionate (1-phenylethyl propanoate); Sylkolide ((E)-2-((3,5-dimethylhex-3-en-2-yl)oxy)-2-methylpropyl cyclopropanecarboxylate); Syvertal (2-(heptan-3-yl)-1,3-dioxolane); Tanaisone ((Z)-1-(cyclooct-3-en-1-yl)ethanone); Tangerinol ((E)-6,10-dimethylundeca-5,9-dien-2-yl acetate); Terpinolene (1-methyl-4-(propan-2-ylidene)cyclohex-1-ene); Terpinyl Acetate (2-(4-methyl-1-cyclohex-3-enyl)propan-2-yl acetate); Terpinyl Formate (2-(4-methyl-1-cyclohex-3-enyl)propan-2-yl formate); Tetra hydro Linalyl Acetate (3,7-dimethyloctan-3-yl acetate); Tetrascone (1-(4,4-dimethyl-2,3-dihydro-1H-naphthalen-1-yl)propan-1-one); Traseolide (1-(1,1,2,6-tetramethyl-3-propan-2-yl-2,3-dihydroinden-5-yl)ethanone); Tridecene-2-Nitrile ((E)-tridec-2-enenitrile); Veloutone (2,2,5-trimethyl-5-pentylcyclopentanone); Velvione ((Z)-cyclohexadec-5-enone); Veracetone (pent-4-enyl methyl ketone); Verdalia ((3aS,4R,6S,7R,7aR)-6-methoxy-3a,4,5,6,7,7a-hexahydro-1H-4,7-methanoindene); Vetikol Acetate/Corps Rhubarb (4-methyl-4-phenylpentan-2-yl acetate); Vetynal ((2R,5R,8S)-4,4,8-trimethyltricyclo[6.3.1.02,5]dodecan-1-yl acetate); Zinarine (2-(2,4-dimethylcyclohexyl)pyridine); and mixtures thereof.

Other odourant ingredients that can be employed in products of the present invention may also be selected from the group consisting of Allogal Base; Armoise Oil Morocco; Citronella Oil Java; Citronella Terpenes; Clary Sage Oil; Clove Leaf Oil; Eucalyptus Citriodora Oil; Fennel Oil; Grapefruit Oil; Lavandin Grosso Oil; Lemongrass Oil; Litsea Cubeba Oil; Armoise Oil Morocco High Thujone; Bergamot Oil; Camphor Oil; Cassis Base; Cedarwood Oil; Celery Seed Oil; Cinnamon Leaf Oil; Citronella Oil; Clary Sage Oil; Cucumber Base; Fir Oil; Galbanum Artessence Oil; Galbanum Oil; Grapefruit Terpenes; Nutmeg Oil; Olibanum Baumarome; Patchouli Oil; Prunal Base; Rhodinol Fraction Ex Geranium; Rosemary Base; Tamarine Base; Tea Tree Oil; Thyme Oil; Ylang Ylang Oil; Vanilla; or mixture thereof.

Although the Michael acceptor compounds, both non-odourant and odourant, referred to hereinabove can be used in products according to the present invention as malodour-counteracting agents, the applicant observed that whereas the Michael acceptors are capable of binding to malodour-causing thiols and trapping them in the form of adducts, depending upon the strength of the Michael acceptor-thiol interaction, the adducts can degrade thermally or oxidatively over time to release the thiol compounds and create a lingering characteristic sulphurous malodour.

For example, products containing the odourant molecule Hedione, in particular, were found to be frequently associated with the sulphurous malodour. Hedione, a widely used odourant ingredient in many perfumery and flavour applications, contains 2-pentylcyclopent-2-en-1-one as an impurity. This impurity can act as a Michael acceptor to trap thiol compounds, and in particular prenylthiol, to form an adduct (see below), from which it can be slowly released over time. Prenyl thiol can also be trapped and subsequently released slowly over time by other Michael acceptor odourant ingredients such as ionones and damascones. Adducts of 2-pentylcyclopent-2-en-1-one (left) and a damascone (right) are shown below:

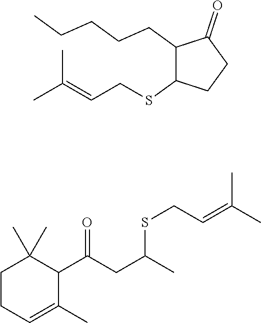 [SMILES: CCCCCC1C(=O)CCC1SCC=C(C)C ; CC(C)=CCSC(C)CC(=O)C1C(C)=CCCC1(C)C]

The observation that hedione-containing products appear to be particularly susceptible to the development of sulphurous malodour could also be a consequence of the fact that hedione is commonly used in large dosages in perfumery and flavour applications, and the use of this ingredient in large dosages in application, together with its physical properties, might push thiol compounds into the headspace and boost their negative sensorial effect.

This theory could also account for an observation that off-notes are often observed too in products, and particularly products used in perfumery and flavours, comprising ester odourant ingredients, which are ubiquitous in large dosages in perfumery and flavour compositions. Such esters include, but are not limited to Agrumex (2-(tert-butyl)cyclohexyl acetate); Allyl Heptanoate (prop-2-enyl heptanoate); Allyl Heptanoate (prop-2-enyl heptanoate); Amyl Salicylate (pentyl 2-hydroxybenzoate); Benzyl Acetate (benzyl acetate); Benzyl Phenyl Acetate (benzyl 2-phenylacetate); Benzyl Salicylate (benzyl 2-hydroxybenzoate); Bornyl Acetate ((25,45)-1,7,7-trimethylbicyclo[2.2.1]heptan-2-yl acetate); Cedryl Acetate ((1S,6R,8aR)-1,4,4,6-tetramethyloctahydro-1H-5,8a-methanoazulen-6-yl acetate); Citronellyl Acetate (3,7-dimethyloct-6-en-1-yl acetate); Cyclohexyl Salicylate (cyclohexyl 2-hydroxybenzoate); Dimethyl Benzyl Carbinyl Acetate (2-methyl-1-phenylpropan-2-yl acetate); Dimethyl Benzyl Carbinyl Butyrate (2-methyl-1-phenylpropan-2-yl butanoate); Ethyl Acetoacetate (ethyl 3-oxobutanoate); Ethyl Butyrate (ethyl butanoate); Ethyl Safranate (ethyl 2,6,6-trimethylcyclohexa-1,3-diene-1-carboxylate); Ethyl-2-Methyl Butyrate (ethyl 2-methylbutanoate); Eugenyl Acetate (4-allyl-2-methoxyphenyl acetate); Floralate ((2,4-dimethylcyclohex-3-en-1-yl)methyl acetate); Florocyclene ((3aR,6S,7aS)-3a,4,5,6,7,7a-hexahydro-1H-4,7-methanoinden-6-yl propanoate); Fraistone (ethyl 2-(2,4-dimethyl-1,3-dioxolan-2-yl)acetate); Fruitate ((3aS,4S,7R,7aS)-ethyl octahydro-1H-4,7-methanoindene-3a-carboxylate); Geranyl Acetate ((E)-3,7-dimethylocta-2,6-dien-1-yl acetate); Hedione (methyl 3-oxo-2-pentylcyclopentaneacetate); Helvetolide ([2-[1-(3,3-dimethylcyclohexyl)ethoxy]-2-methylpropyl]propanoate); Hexenyl-3-Cis Acetate ((Z)-hex-3-en-1-yl acetate); Hexenyl-3-Cis Salicylate ((Z)-hex-3-en-1-yl 2-hydroxybenzoate); Hexyl Acetate (hexyl acetate); Hexyl Salicylate (hexyl 2-hydroxybenzoate); Isononanyl Acetate (3,5,5-trimethylhexyl acetate); Jasmacyclene ((3aR,6S,7aS)-3a,4,5,6,7,7a-hexahydro-1H-4,7-methanoinden-6-yl acetate); Linalyl Acetate (3,7-dimethylocta-1,6-dien-3-yl acetate); Manzanate (ethyl 2-methylpentanoate); Nerolidyle ((Z)-3,7,11-trimethyldodeca-1,6,10-trien-3-yl acetate); Para Tert Butyl Cyclohexyl Acetate (4-(tert-butyl)cyclohexyl acetate); Vera moss (methyl 2,4-dihydroxy-3,6-dimethylbenzoate); Vetiveryl Acetate ((4,8-dimethyl-2-propan-2-ylidene-3,3a,4,5,6,8a-hexahydro-1H-azulen-6-yl) acetate); and mixtures thereof.

Accordingly, in particular embodiments of the invention, products comprising a bio-sourced ingredient containing ocimene; an ingredient that can act, or contains impurities that can act, as a Michael acceptor, such as hedione, damascones or ionones, or any of the ester odourant ingredients referred to hereinabove, should preferably also employ one of the other means for suppressing or eliminating malodour described herein. In particular, products containing mixtures of these ingredients are preferably treated with an oxidizing agent, and still more particularly, the container is pre-treated with an oxidizing agent, as more fully described hereinabove.

Another means of malodour-counteraction is to control the level of water present in a product. This can be particularly effective in regards to products that are hydro-alcoholic, such as our found in fine perfumery. In fine perfumery, it is conventional to use hydro-alcoholic solvents, such as ethanol/water mixtures that can contain water levels as high as 15 wt %.

In accordance with the present invention there is provided a method of suppressing or eliminating a malodour in a fine perfumery product containing a bio-sourced ingredient and a container for holding and/or dispensing the ingredient, wherein the bio-sourced ingredient contains ocimene and wherein the method comprises the step of limiting the amount of water in the product to less than 3 wt %, and more particularly to 2 wt % or less.

The invention also provides in another of its aspects a fine perfumery product, comprising at least one perfume ingredient, a hydroalcoholic solvent, such as a water/ethanol mixture, a bio-sourced ingredient comprising ocimene, and a container for holding and/or dispensing the same, wherein the product comprises less than 3 wt %, and more particularly to 2 wt % or less of water.

The invention will be further explained and illustrated with reference to the following non-limiting examples.

### Example 1

### Preparation of Perfume Samples

A model perfume was prepared by dissolving 1% (w/w) Hedione (methyl dihydrojasmonate—obtained from Firmenich SA) in ethanol/water (85%/15% w/w) and labeled ‘Perfume A’. ‘Perfume A’ was treated with either 0.04% (w/w) ocimene (mixture of isomers obtained from International Flavors and Fragrances), 0.04% (w/w) ‘Lavender Oil France’ (obtained from Givaudan SA), 0.04% (w/w) myrcene (obtained from Sigma-Aldrich), 0.04% of a stock solution of prenylthiol (CAS 5287-45-6, purchased e.g. from Sigma Aldrich) in Ethanol (concentration 25 ppm), or no additive. Samples were transferred into glass bottles and either closed with sprays containing different gasket types (see table below) or closed with a screw cap not containing vulcanized rubber gaskets. This resulted in the following perfume samples shown in Table 1, which were stored at 60° C. in an oven, for 7 days. Storage at elevated temperature was used to accelerate and accentuate the formation of the off-note. Samples stored at room temperature showed similar results but required longer storage times.

Gaskets made of different materials were obtained from Aptar Group Inc.: BUNA (Butadiene-acrylonitrile copolymer—vulcanized); Butyl (Isobutylene-Isoprene copolymer—vulcanized); ENB (ethylidene norbornene—vulcanized).

PE (polyethylene—not vulcanized) gasket was obtained from Gravis Orly.

TABLE 1

| Sample Number | Additive (0.04% w/w) | gasket |
|---|---|---|
| 1 | Ocimene | none |
| 2 | Ocimene | Butyl |
| 3 | Ocimene | Buna + ENB |
| 4 | Ocimene | PE |
| 5 | Lavender Oil France | none |
| 6 | Lavender Oil France | Butyl |
| 7 | Lavender Oil France | Buna + ENB |
| 8 | Myrcene | Butyl |
| 9 | none | Butyl |
| 10 | Prenylthiol | none |
| | (25 ppm in Ethanol) | |

### Analytical Techniques

### 1) GC-Sniffing

One microliter aliquots of the samples from Table 1 were manually injected and analyzed on a mass spectrometer Agilent 59773 GC/MSD connected to a gas chromatograph Agilent 8890 GC System equipped with a split injector, an apolar HP-1MS capillary column (60 m, 0.32 mm internal diameter (i.d.), 0.25 μm film thickness) and a sniffing port (Givaudan in-house product). The carrier gas was helium, and the flow rate was set at 3.5 ml/min (constant flow mode). The split injector was maintained at 250° C., and the split ratio was 10:1. The oven temperature was programmed to rise from 70° C. to 97° C. at 3° C./min, then to 270° C. at 40° C./min and held for 7 min. At the end of the capillary column, the effluent was split between the sniffing port and the MSD using deactivated fused silica capillaries (1.5 m, 0.15 mm i.d. to sniffing port and 0.65 m, 0.1 mm i.d. to MSD). Mass spectra were generated in electron ionization (El) full scan mode (m/z range 30-450, 70 eV). Compound identification was made by comparing odour qualities, mass spectra and retention times with those of the respective reference compounds. Sensory evaluations at the sniffing port were performed by a trained panel of seven assessors.

### 2) GC-MS/MS

Aliquots (500 μl) of samples from Table 1 were dried by adding 1000 111 of MtBE and ^{˜}450 mg MgSO₄ followed by filtration through a Na₂SO₄ cartridge. 1 μl of the dried sample was injected in a deactivated glass wool equipped liner ata split ratio of 1:10 using an inlet temperature of 230° C. The GC-MS (Thermo Trace 1310 Series/Thermo TSCA. 8000) was equipped with an Agilent J&W HP-5MS capillary column (15 m, 0.25 mm i.d. 0.25 μm film) operated at a constant He flow of 2 ml/min. The temperature program started at 35° C. (for 2 min), then with 20° C./min to 75° C. followed by 40° C./min to 270° C. (for 3 min). The MS transfer line and ion source temperatures were 250° C. and 200° C., respectively. The triple quadrupole MS was equipped with an El ion source at 70 eV. An SRM detection method was used using the precursor ion mass of 102, collision energy of 10 and fragment ion mass of 69. Data was analyzed using Thermo Xcalibur 4.2.28.14.

### 3) Blotter Assessment

Blotter assessment of samples was conducted by dipping a paper blotter into the solution and allowing it to dry in the air for 5 seconds. The blotters were then smelled by a panel of three expert perfumers and characterized by the presence or absence of the characteristic prenylthiol off-note. In all cases tested, results from blotter evaluation were consistent with those observed in GC-Sniff analysis.

### Results

The samples described in Table 1 were analyzed using the Methods described above. The results are summarized in Table 2:

TABLE 2

| | off note perceived | off note perceived | prenylthiol |
|---|---|---|---|
| Sample | in sensory* | in GC-Sniff* | detected** |
| 9 | no | no | no |
| 1 | no | no | no |
| 2 | yes | yes | yes |
| 10 | yes | yes | yes |
| 8 | no | not determined | not determined |
| 4 | no | not determined | not determined |
| 3 | yes | not determined | not determined |
| 5 | no | not determined | not determined |
| 6 | yes | not determined | not determined |
| 7 | yes | not determined | not determined |

*sensory by panel form blotter and/or by GC-Sniff
**detected by GC-Sniff and GC-EI MS/MS

### Example 2

### Preparation of Perfume Samples

A model perfume was prepared by dissolving 1% (w/w) Hedione (methyl dihydrojasmonate—obtained from Firmenich SA) and 0.04% (w/w) ocimene (obtained from International Flavors and Fragrances) in ethanol/water (85%/15% w/w) and labeled ‘Perfume B’. ‘Perfume C’ was prepared by dissolving 70 ppb (parts per billion w/w) of prenylthiol (CAS 5287-45-6, purchased e.g. from Sigma Aldrich) in ‘Perfume B’. Finally, ‘Perfume C’ was treated with 0.1% (w/w) dimethylfumarate (CAS 624-49-7 purchased e.g. from Sigma-Aldrich) to give ‘Perfume D’.

### Blotter Assessment

Blotter assessment of Perfumes B, C and D was conducted by dipping paper blotters into each solution and allowing them to dry in the air for 5 seconds. The blotters were then smelled by a panel of five expert assessors and characterized by the presence or absence of the characteristic prenylthiol off-note, by consensus of the five panellists.

### Results

The Perfumes B, C and D described above were analyzed by blotter assessment. The results are summarized in Table 3:

TABLE 3

| Sample | Odour description |
|---|---|
| Perfume B | light green floral; no off-note. |
| Perfume C | clear off-note; unpleasant, beer, fermented. |
| Perfume D | no off-note; not distinguishable from Perfume B. |

### CONCLUSION

The addition of dimethyl fumarate to a perfume containing the prenylthiol off-note acts as an efficient scavenger, removing the off-odor without otherwise disturbing the composition.

### Example 3

Malodour-Reduction: Effect of Pre-Treating a Container with Hydrogen Peroxide

An atomizer pump was attached to a perfume flask (total volume ca. 5 mL) containing 5 mL 35% hydrogen peroxide and the pump was pressed to spray hydrogen peroxide through it until half the content was dispensed (ca. 20× pressing).

Olfactive assessment of the hydrogen peroxide alone didn't reveal any off-notes. During pumping through the atomizer, the efflux was captured on blotter and evaluated. A pervasive sulphurous/acidic off-note was detected, indicating that the pump forms an off-note when in contact with hydrogen peroxide under the conditions of the experiment. The off-note could also be smelled in the air while operating the pump to dispense hydrogen peroxide.

A cap was attached to the perfume flask containing ca. 2.5 mL of 35% hydrogen peroxide, and the flask was allowed to sit for 24 h at room temperature.

After storage, the rest of the hydrogen peroxide solution was dispensed through the pump (pressing c. 20×).

The caps were then briefly rinsed with water on the outside and then thoroughly purged/rinsed by pressing 10 mL water through the pump in the same way as above, followed by 5 mL Ethanol in the same way.

Olfactive assessment of the water and ethanol purges was performed by spraying on blotter in the manner described above. No off-notes were detected in any of the two, showing that the cause of the off-note can be easily rinsed off.

The caps were pumped dry (ca. 10×) and then allowed to dry under air.

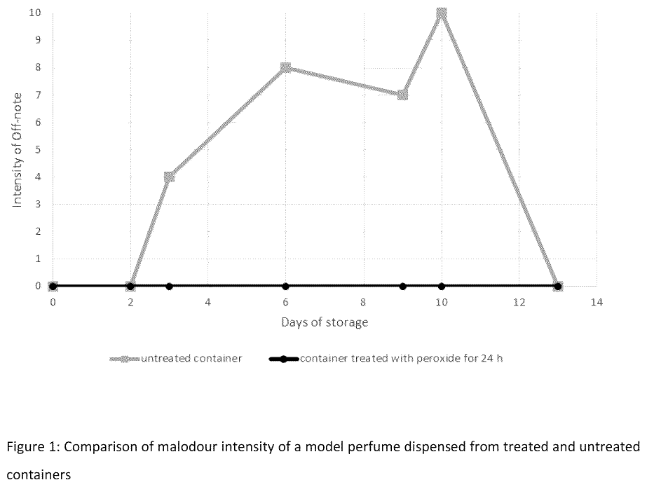

Perfume flasks were filled with a model perfume. Test flasks were equipped with pumps treated with peroxide in the manner described above, and for comparative purposes, the same model perfume was filled into flasks equipped with untreated pumps. The perfumes from both test and comparative pumps were dispensed onto blotters on consecutive days and subjected to olfactive assessment. The results are visualized in FIG. 1. As can be seen in FIG. 1 significant malodour emanates from the perfume dispensed from the untreated pump for a number of days. In contrast, no malodour could be detected on perfume dispensed from the treated pump.

1. A method of suppressing or eliminating a malodour from a product, the product comprising a bio-sourced ingredient and a container for holding, dispensing, or holding and dispensing the ingredient, wherein the bio-sourced ingredient contains ocimene and wherein the method comprises at least one of the following steps:
preventing contact of ocimene with material in the container capable of reacting with ocimene to generate a thiol compound associated with a sulphurous malodour;
removing from the product any thiol compound generated as the result of the reaction of ocimene with a co-reactant in the container; or
treating the product with a malodour-counteracting agent.

2. A method according to claim 1, wherein the product is selected from the group consisting of a perfume, cosmetic, a flavour, a food or beverage product, a product delivering a health & wellness benefit, a nutraceutical product, a pharmaceutical product, or a product comprising a functional ingredient selected from a preservative, an anti-oxidant, an anti-microbial, a dye or pigment, a texturizer or an emulsifier.

3. A method according to claim 1, wherein the bio-sourced ingredient is selected from the group consisting of ocimene, Tagete Oil; Oppoponax Oil; Neroli Oil; Lavender Oil; Angelica Root Oil; Lavandin Abrialis Oil; Angelica Seed Oil; Lime Oxide; Fennel Oil; Menthe Citrate; Petitgrain Oil; Basil Oil; Lentisque Oil; Mint Oils, Lime Oil; Lemon Oil; Orange Oil; Mandarin Oil; Ho Leaf; Hops; Kumquat; Mango; Bigarde; Parsley Oil; Asafoetida Oil; Ay Oil; Basil Oil; Bay Oil; Bergamot Oil; Black Pepper Oil; Buchu Oil; Cananga Oil; Carrot Seed Oil; Celery Seed Oil; Chamomile Oil; Cinnamon Bark Oil; Clary Sage Oil; Coriander Seed Oil; Cubebe Oil; Davana Oil; Dill Seed Oil; Estragon Oil; Eucalyptus Oil; Fennel Oil; Geraniol Oil; Ginger Oil; Grapefruit Oil; Jasmin Absolute; Laurel Leaf Oil; Lemon Oil; Lime Oil; Lovage Root Oil; Marjoram Oil; Neroli Oil; Niaouli Oil; Nutmeg Oil; Oil Of Labdanum; Orange Flower Oil; Palmarosa Oil; Pennyroyal Oil; Peppermint Terpenes; Petitgrain Mandarine Oil; Pine Needle Oil; Cardamom Oil; Rosemary Oil; Shiso Oil; Spearmint Oil; Star Anise Oil; Valerian Oil; Vanilla Extract; and mixtures thereof.

4. A method according to claim 1, wherein the thiol compound is prenylthiol or 2-methyl-3-furanthiol.

5. A method according to claim 1, wherein the product is treated with a malodour-counteracting agent that is an oxidizing agent.

6. A method according to claim 5, wherein the oxidizing agent is selected from the group consisting of hydrogen peroxide, organic peroxides, inorganic peroxides, peroxide salts, including all of their hydrated forms; oxidized halogen salts; oxidizing gases; per-acids, and oxidizing enzyme systems.

7. A method according to claim 5, wherein the container is pre-treated with the oxidizing agent before the bio-sourced material is brought into contact with the container.

8. A method according to claim 6, wherein the oxidizing agent is hydrogen peroxide.

9. A product comprising a bio-sourced ingredient and a container for holding, dispensing, or holding and dispensing the ingredient, wherein the bio-sourced ingredient contains ocimene, and wherein the product is treated with an oxidizing agent.

10. A product according to claim 9, wherein the treatment with an oxidizing agent comprises the pre-treatment of the container with the oxidizing agent.

11. A product according to claim 9, wherein the container, or part of the container comprises a sulphur-containing vulcanized polymer or sulphur-containing vulcanizing agent.

12. A product according to claim 9, which is a fine perfumery product comprising a perfume containing ocimene-containing bio-sourced ingredient.

13. A product according to claim 12, wherein the perfume contains a perfume ingredient that is a Michael acceptor.

14. A product according to claim 12, wherein the perfume comprises a hydro-alcoholic solvent optionally having a water content of less than 3 wt %, and more particularly 2 wt % or less.

15. A container for holding, dispensing, or holding and dispensing a product as defined in claim 9, treated with an oxidizing agent.

16. A product according to claim 10, wherein the container, or part of the container comprises a sulphur-containing vulcanized polymer or sulphur-containing vulcanizing agent.

17. The container according to claim 15, wherein the oxidizing agent is selected from the group consisting of hydrogen peroxide, organic peroxides, inorganic peroxides, peroxide salts, including all of their hydrated forms; oxidized halogen salts; oxidizing gases; per-acids, and oxidizing enzyme systems.

18. The container according to claim 17, wherein the oxidizing agent is hydrogen peroxide.

19. A product according to claim 13, wherein the perfume comprises a hydro-alcoholic solvent optionally having a water content of less than 3 wt %.

20. The container according to claim 15, wherein the container, or part of the container comprises a sulphur-containing vulcanized polymer or sulphur-containing vulcanizing agent.

21. The container according to claim 17, wherein the container, or part of the container comprises a sulphur-containing vulcanized polymer or sulphur-containing vulcanizing agent.

22. The container according to claim 18, wherein the container, or part of the container comprises a sulphur-containing vulcanized polymer or sulphur-containing vulcanizing agent.
